(19) United States
(12) Patent Application Publication
(10) Pub. No.: US 2024/0013871 A1
(43) Pub. Date: Jan. 11, 2024

(54) SAAS PLATFORM TO MANAGE CLINICAL TRIAL

(71) Applicants: Vivek Asthana, Meerut (IN); Ankur Agrawal, Gurugram (IN)

(72) Inventors: Vivek Asthana, Meerut (IN); Ankur Agrawal, Gurugram (IN)

(21) Appl. No.: 18/043,164

(22) PCT Filed: Aug. 27, 2021
(86) PCT No.: PCT/IB2021/057849

§ 371 Date: Feb. 27, 2023

(30) Foreign Application Priority Data
Aug. 28, 2020 (IN) 202041037119

(51) Int. Cl. G16H 10/20; G16H 20/00; G16H 10/60

(52) U.S. Cl. CPC G16H 10/20; G16H 20/00; G16H 10/60

(57) ABSTRACT

A SaaS platform to manage a clinical trial is disclosed. The platform enables on-boarding of patients for the clinical trial for one or more clinical research organizations. Such details are necessary for categorisation of the one or more patients. A medical compliance module is set up to capture real time bio-physical parameters indicative of health condition of the patients. The medical compliance module also monitors adherence of each of the patients to the one or more treatment plans designed for the clinical trials. Additionally, a database management module receives, and stores data associated with the clinical trial from the one or more clinical research organisations. Storing of such classified data is being done with specific privacy level for security. An access control module further enables collaborative clinical trial among the one or more clinical research organisations by sharing the data associated with the clinical trial in real-time.

### EARLIEST PRIORITY DATE

This Application claims priority from a complete patent application filed in India having Patent Application No. 202041037119, filed on Aug. 28, 2020, and titled “SAAS PLATFORM TO MANAGE CLINICAL TRIAL” and claims priority from a PCT patent application having Patent Application No. PCT/IB2021/057849, filed on Aug. 27, 2021, and titled “SAAS PLATFORM TO MANAGE CLINICAL TRIAL”.

### FIELD OF INVENTION

Embodiments of a present disclosure relates to a field of healthcare, and more particularly to a SaaS platform to manage a clinical trial for clinical research organizations.

### BACKGROUND

A vital step in healthcare decision making is to generate a high-quality medical data through clinical trials. The clinical trial process mainly helps in finding improved ways to prevent, screen for, diagnose, or treat any disease. Clinical trial may be stated as a study which follows strict scientific standards to identify new drugs and treatment plans while protecting patients participating in the clinical trials.

Any clinical trial may be divided into multiple phases, where each phase has a different purpose and help researchers answer different questions. Mainly in each phases a large number of patients are administered with an experimental drug to evaluate drug safety, possible drug side effects and efficacy. Additionally, clinical trial also determines how the drug should be used or delivered.

In conventional approach, clinical trial data corresponding to large number of patients is handled manually. Here, the medical appropriated data is maintained manually by researchers and medical personnel. Further, monitoring of the patients under observation is also done manually.

Such manual monitoring process often leads to error and confusion. Additionally, at one instance multiple clinical trials takes place for any specific disease, it is vital to bring all such trials under one platform to achieve a better result in handling the disease.

The manual aspect of the management of medical data associated with clinical trials prevents multiple research institutions to collaborate in real time. This being a big hindrance in scaling up of the clinical trials.

Another issue in some of the present system of clinical trials is that all the patients need to be located at a one place during the trials. Since, there are no efficient tools to integrate the medical data from multiple sources and process the same.

Hence, there is a need for an improved platform to manage a clinical trial corresponding to one or more clinical research organizations and a method to operate the same and therefore address the aforementioned issues.

### BRIEF DESCRIPTION

In accordance with one embodiment of the disclosure, a SaaS platform to manage a clinical trial is disclosed. The platform includes a clinical trial registration module. The clinical trial registration module is hosted in a presentation tier of the SaaS platform. The clinical trial registration module is configured to on-board one or more patients for the clinical trial for one or more clinical research organizations based at least one of name, age, contact details, address, medical history family history, genetic data, nutritional data or combination thereof. The clinical trial registration module is also configured to categorise on-boarded one or more patients based on age and health condition.

The platform includes a medical compliance module. The medical compliance module is hosted in a logic tier of the SaaS platform. The medical compliance module is configured to capture data representative of at least one of one or more diseases, one or more bio-physical parameters indicative of health condition of the one or more patients, type of discomfort, severity of discomfort, part of body experiencing the discomfort, time of discomfort, one or more treatment plans designed for the clinical trials, one or more medications prescribed in accordance with the one or more treatment plans, one or more symptoms, one or more medication side-effects via communicatively coupled one or more devices associated with at least one of each of the one or more patients, one or more healthcare personnel and one or more doctors.

The medical compliance module is also configured to monitor adherence of each of the one or more patients to the one or more treatment plans designed for the clinical trials, prescribed follow-ups and actions. The medical compliance module is also configured to generate a medical adherence report corresponding to each of the one or more patients in compliance with the one or more treatment plans designed for the clinical trials.

The platform also includes a database management module. The database management module is hosted in a data tier of the SaaS platform. The database management module is configured to automatically store data captured by the medical compliance module and generated medical adherence report for each of the one or more patients. The database management module is also configured to receive and store data associated with the clinical trial from the one or more clinical research organisations based on one of data migration, data conversion and data integration techniques. The database management module is also configured to classify stored data associated with the clinical trial based on predesigned privacy levels.

The platform also includes an access control module. The access control module is hosted in the in the logic tier of the SaaS platform. The access control module is configured to provide access to a specific privacy level of classified stored data associated with the clinical trial corresponding to the one or more clinical research organisations based on a permitted access to a specific privacy level. The access control module is configured to enable collaborative clinical trial among the one or more clinical research organisations by sharing the data associated with the clinical trial in real-time.

In accordance with one embodiment of the disclosure, a method for managing a clinical trial on a SaaS platform is disclosed. The method includes on-boarding one or more patients for the clinical trial for one or more clinical research organizations based at least one of name, age, contact details, address, medical history, family history, genetic data, nutritional data or combination thereof. The method also includes categorizing on-boarded one or more patients based on age and health condition. The method also includes capturing data representative of at least one of one or more diseases, one or more bio-physical parameters indicative of health condition of the one or more patients, type of discomfort, severity of discomfort, part of body experiencing the discomfort and time of discomfort, one or more treatment plans designed for the clinical trials, one or more medications prescribed in accordance with the one or more treatment plans, one or more symptoms, one or more medication side-effects via communicatively coupled one or more devices associated with at least one of each of the one or more patients, one or more healthcare personnel and one or more doctors.

The method also includes monitoring adherence of each of the one or more patients to the one or more treatment plans designed for the clinical trials, prescribed follow-ups and actions. The method also includes generating a medical adherence report corresponding to each of the one or more patients in compliance with the one or more treatment plans designed for the clinical trials. The method also includes storing automatically data captured by the medical compliance module and generated medical adherence report for each of the one or more patients.

The method also includes receiving and storing data associated with the clinical trial from the one or more clinical research organisations based on one of data migration, data conversion and data integration techniques. The method also includes classifying stored data associated with the clinical trial based on predesigned privacy levels. The method also includes providing access to a specific privacy level of classified stored data associated with the clinical trial corresponding to the one or more clinical research organisations based on a permitted access to a specific privacy level. The method also includes enabling collaborative clinical trial among the one or more clinical research organisations by sharing the data associated with the clinical trial in real-time.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram representation of a SaaS platform to manage a clinical trial in accordance with an embodiment of the present disclosure;

FIG. 2 is a schematic representation of an embodiment representing input mechanism window related to disease corresponding to a registered patient of FIG. 1 in accordance with an embodiment of the present disclosure;

FIG. 3 is a schematic representation of an embodiment representing the adherence dashboard window corresponding to the registered patient of FIG. 1 in accordance with an embodiment of the present disclosure;

FIG. 4 is a schematic representation of an embodiment representing the adherence dashboard window with real time information corresponding to the registered patient of FIG. 1 in accordance with an embodiment of the present disclosure.

FIG. 5 is a schematic representation of an embodiment representing the SaaS platform to manage a clinical trial of FIG. 1 in accordance with an embodiment of the present disclosure;

FIG. 6 is a block diagram of a computer or a server in accordance with an embodiment of the present disclosure;

FIG. 7 is a flowchart representing the steps of a method for managing a clinical trial on a SaaS platform in accordance with an embodiment of the present disclosure;

FIG. 8 is a flowchart representing the steps of a method for managing a clinical trial on a SaaS platform in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated online platform, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or subsystems or elements or structures or components preceded by “comprises . . . a” does not, without more constraints, preclude the existence of other devices, subsystems, elements, structures, components, additional devices, additional subsystems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relates to a SaaS platform to manage a clinical trial. The platform enables on-boarding of one or more patients for the clinical trial for one or more clinical research organizations based on at least one of name, age, contact details, address, medical history, family history, genetic data, nutritional data or combination thereof. Such details are necessary for categorisation of the one or more patients.

A medical compliance module in logic tier of the platform, is set up to capture real time bio-physical parameters indicative of health condition of the one or more patients, type of discomfort, severity of discomfort, one or more symptoms, one or more medication side-effects and the like. The medical compliance module monitors adherence of each of the one or more patients to the one or more treatment plans designed for the clinical trials, prescribed follow-ups and actions. A medical adherence report corresponding to each of the one or more patients in compliance with the one or more treatment plans is created and stored for the clinical trials.

Additionally, a database management module receives, and stores data associated with the clinical trial from the one or more clinical research organisations based on one of data migration, data conversion and data integration techniques. Storing of such classified data is being done with specific privacy level for security. An access control module further enables collaborative clinical trial among the one or more clinical research organisations by sharing the data associated with the clinical trial in real-time.

A computer system (standalone, client or server computer system) configured by an application may constitute a “platform” or “module” that is configured and operated to perform certain operations. In one embodiment, the “platform” or “module” may be implemented mechanically or electronically, so a platform or module may comprise dedicated circuitry or logic that is permanently configured (within a special-purpose processor) to perform certain operations. In another embodiment, a “platform” or “module” may also comprise programmable logic or circuitry (as encompassed within a general-purpose processor or other programmable processor) that is temporarily configured by software to perform certain operations.

Accordingly, the term “module” or “platform” should be understood to encompass a tangible entity, be that an entity that is physically constructed permanently configured (hardwired) or temporarily configured (programmed) to operate in a certain manner and/or to perform certain operations described herein.

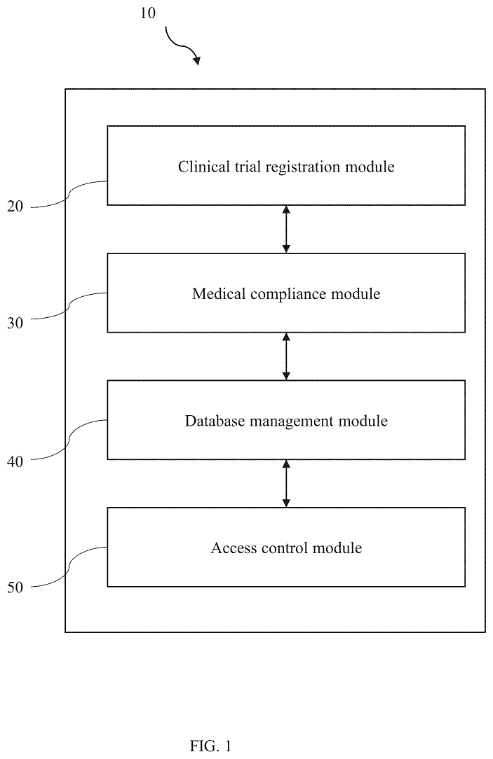

FIG. 1 is a block diagram representation of a SaaS platform 10 to manage a clinical trial in accordance with an embodiment of the present disclosure. As used herein, the term “Software as a service (SaaS)” is a software licensing and delivery model in which software is licensed on a subscription basis and is centrally hosted. In one embodiment, the SaaS platform comprises one of a multi-tenant SaaS platform and single-tenant SaaS platform. As used herein, “tenant” refers to a group of users who share a common access with specific privileges to the software instance.

It is pertinent to note, that the platform 10 enables real-time interactions between patients and clinical research organizations for constant monitoring. Such constant monitoring is very much important for discovering new treatments for diseases, as well as new ways to detect and diagnose the diseases.

The platform 10 includes a clinical trial registration module 20. The clinical trial registration module 20 is hosted in a presentation tier of the SaaS platform 10. The clinical trial registration module 20 is configured to on-board one or more patients for the clinical trial for one or more clinical research organizations. In one embodiment, during registration process the one or more patients provide information such as name, age, contact details, address, medical history, family history, genetic data, nutritional data and the like.

In another embodiment, the one or more clinical research organizations comprises government agencies, private agencies, pharmaceutical companies, and institutes. In such embodiment, the clinical research organization supports drug manufacturers on their process to discover and approve drugs of the future by absorbing some of the clinical stage burdens. Here, the clinical stage burdens may be manually monitoring of patients.

After on-boarding process, the registered information is categorised based on age and health condition. For the safety and effectiveness (efficacy) of medications, devices, diagnostic products and treatment regimens various parameters are taken into consideration during the clinical trial. For example, for COVID 19 clinical trial the age and health comorbidities are taken into consideration. Such vital information is required to understand how the medication are behaving with particular age group or with comorbidities.

The platform 10 also includes a medical compliance module 30. The medical compliance module 30 is hosted in a logic tier of the SaaS platform 10. The medical compliance module 30 is configured to capture data representative of at least one of one or more diseases, one or more bio-physical parameters indicative of health condition of the one or more patients, type of discomfort, severity of discomfort, part of body experiencing the discomfort, time of discomfort, one or more treatment plans designed for the clinical trials, one or more medications prescribed in accordance with the one or more treatment plans, one or more symptoms, one or more medication side-effects via communicatively coupled one or more devices associated with at least one of each of the one or more patients, one or more healthcare personnel and one or more doctors.

It is pertinent to note that the data corresponding to the each of the one or more patient's health condition is captured directly from the doctor and healthcare personnel devices or computing system. The one or more healthcare personnel may include allied healthcare personnel, geriatric care personnel and the like.

In one embodiment, the platform captures data representative of at least one of one or more diseases via at least one of textual input, pre-designated field input and input on 3D image representative of human body. In one embodiment, the provided texts are interpreted with data extraction techniques to examine deeper. Hence, textual inputs are classified and extracted to relevant information in a structured manner. In another embodiment, the patient may also provide data via the voice input mechanism.

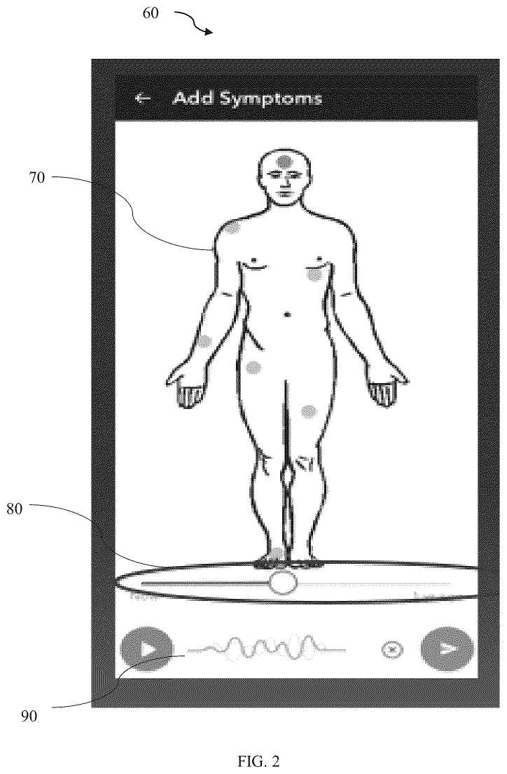

FIG. 2 is a schematic representation of an embodiment representing input mechanism window 60 related to disease corresponding to a registered patient of FIG. 1 in accordance with an embodiment of the present disclosure. A text or voice input bar 90 is located at the bottom end of the recording input window 60. It is pertinent to note that each patient may also provide voice input as well as pictorial input via the text or voice input bar 90. Here, the 3D image representative of human body 70 may be maximized or minimised by a slider 90 to provide input.

In another embodiment, the data representative of at least one of the one or more diseases and the one or more bio-physical parameters of the registered patient is captured via IOT enabled one or more wearable sensors. As used herein, the term “disease” refers to a disorder of structure or function in a human, especially one that produces specific symptoms or that affects a specific location and is not simply a direct result of physical injury. The IOT enabled one or more wearable sensors include smart watch, smart bangle and the like. In such embodiment, any bio-physical parameter such as heart rate, body pressure level may be easily be recorded in real time for better tracking.

The medical compliance module 30 is configured to monitor adherence of each of the one or more patients to the one or more treatment plans designed for the clinical trials, prescribed follow-ups and actions. Here, monitoring is basically done on the basis of “YES” or “NO” notification from each of the patient.

In one exemplary embodiment, two registered patients with age 65 is suffering from COVID 19 as well as high blood pressure. Two registered doctors associated with each of the patients are monitoring blood pressure as well as fever constantly. Both doctors for treating uses different fever medication but same blood pressure medicine. Here, the platform 10 generates automatic “YES” or “NO” notifications to understand whether the patients have consumed the medications on time or not. The platform 10 thereby tries to monitor both the patient with different medication. The platform 10 thereby forces the patients to adhere to the medication plan.

The platform 10 monitor adherences of each of the one or more patients to the one or more treatment plans designed for the clinical trials, prescribed follow-ups and actions at a micro level. In one embodiment, the adherence at the micro level comprises monitoring daily medication intakes in accordance with the one or more treatment plans, monitoring follow-up treatment compliance in accordance with the one or more treatment plans, monitoring medical appointments in accordance with the one or more treatment plans and monitoring ongoing symptoms and side-effects.

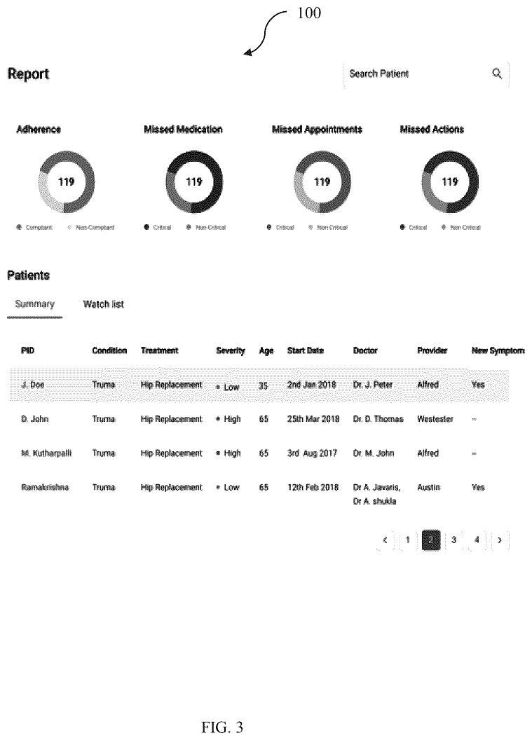

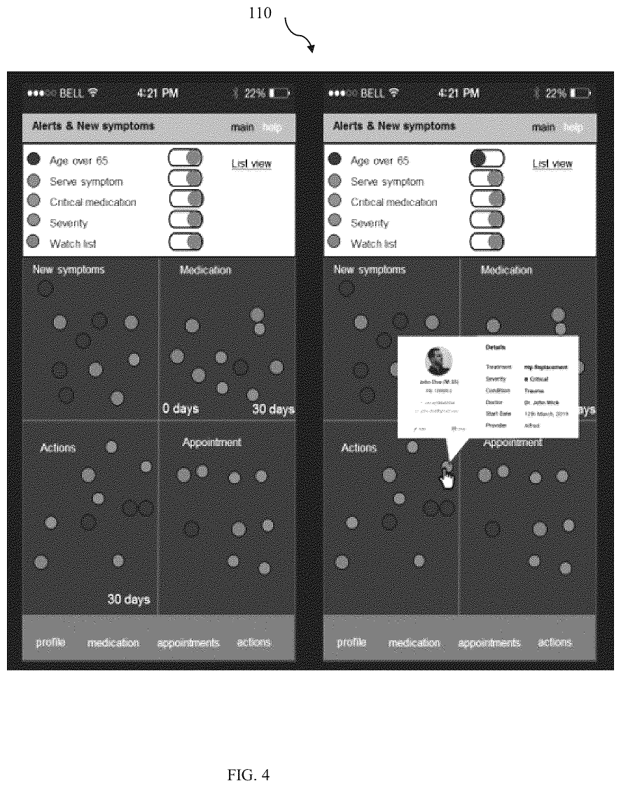

FIG. 3 is a schematic representation of an embodiment representing the adherence dashboard window 100 corresponding to the registered patient of FIG. 1 in accordance with an embodiment of the present disclosure. FIG. 4 is a schematic representation of an embodiment representing the adherence dashboard window 110 with real time information corresponding to the registered patient of FIG. 1 in accordance with an embodiment of the present disclosure.

Clinical trials are basically devised in five phases. The five phases are phase 0, phase I, phase II, phase III and phase IV. In each phase, patients of required number are selected for trials. Through the medical compliance module 30, the selected patients are monitored easily. The medical compliance module 30 enables complete and effective adherence to clinical medical regimes or medical treatment plans.

The medical compliance module 30 is also configured to generate a medical adherence report corresponding to each of the one or more patients in compliance with the one or more treatment plans designed for the clinical trials. In the above stated exemplary embodiment, the platform 10 generates individual report for each patient with respect to administered medication. Such adherence report is very much necessary in clinical trial to diagnose problems or side effects.

The platform 10 also includes a database management module 40. The database management module 40 is hosted in a data tier of the SaaS platform 10. The database management module 40 is configured to automatically store data captured by the medical compliance module and the generated adherence report. Here, the storage may be remote storage or local storage. The database management module 40 is also configured to receive, and store data associated with the clinical trial from the one or more clinical research organisations based on one of data migration, data conversion and data integration techniques.

In one embodiment, the data migration comprises migrating databases, migrating applications and migrating to the cloud. It is pertinent to note that clinical trial records from the one or more clinical research organisations may be of different format. The database management module 40 enables receiving and storing of such different format records. Before storing, the received files are converted to a common format.

The conversion may require processing by the use of a special conversion program, or it may involve a complex process of going through intermediary stages, or involving complex “exporting” and “importing” procedures, which may include converting to and from a tab-delimited or comma-separated text file. In such embodiment, the database management module 40 recognizes recorded file format at the receiving stage and store the recorded file as requested format. Further, upon not recognizing the source format or target format the database management module 40 may convert the recorded file in an intermediate format, which may then be reformatted later.

The database management module 40 is also configured to classify stored data associated with the clinical trial based on predesigned privacy levels. It is pertinent to note that such privacy level is very much required as a doctor should only have access assigned patients adherence report, similarly a specific clinical research organization should have access to specific associated data. Each clinical research organization should take permissions from each of the one or more patients before sharing data with third parties or research organizations.

Additionally, the medical compliance module 30 is also configured to map the one or more treatment plans generated by the one or more clinical research organisation with the one or more treatment plans generated by another clinical research organisation. Here, the platform 10 basically identifies differences in at least one of medications, prescribed follow-ups and actions, one or more symptoms, one or more medication side-effects and health status improvement rate.

After monitoring and real time tracking, the medical compliance module 40 is also configured to suggest standardisation of the clinical trial parameters. The standardisation process is based on identified differences in at least one of medications, prescribed follow-ups and actions, one or more symptoms, one or more medication side-effects and health status improvement rate. It is pertinent to note that the medical compliance module 10 recognizes end results of each treatment plans and compares each result for understanding the best treatment plans.

The medical compliance module 10 also compares blind controls or double-blind randomization with the end results of an ongoing clinical trials and compares analysis of different clinical trials for same disease or medications. The medical compliance module 10 presents the follow-up procedure of the best treatment plan, details regarding the one or more medication side-effects corresponding to the best treatment plan and the like in a structured report format.

The platform 10 also includes an access control module 50. The access control module 50 hosted in the in the logic tier of the SaaS platform 10. The access control module 50 is configured to provide access to a specific privacy level of classified stored data associated with the clinical trial corresponding to the one or more clinical research organisations based on a permitted access to a specific privacy level. Here, each of the one or more patients and the clinical research organization may establish specific required privacy level.

The access control module is also configured to enable collaborative clinical trial among the one or more clinical research organisations by sharing the data associated with the clinical trial in real-time. The database management module 40 allows sharing stored data with respect to associated specific privacy level.

The platform 10 also includes a feedback module. The feedback module is hosted in the in the data tier of the SaaS platform 10. The feedback module is configured to facilitate exchange of real-time feedbacks among the one or more clinical research organisations. In such embodiment, the real-time feedbacks enable constant monitoring of each of the one or more patient's health condition.

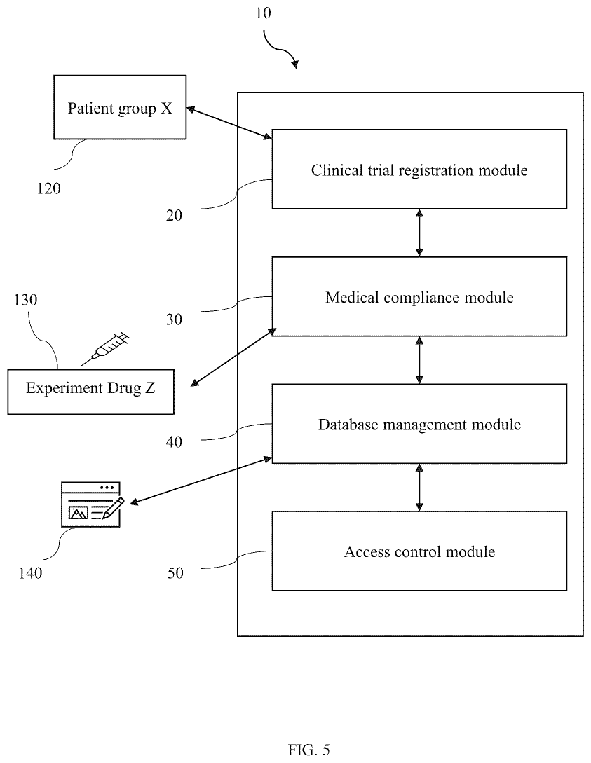

FIG. 5 is a schematic representation of an embodiment representing the SaaS platform 10 to manage a clinical trial of FIG. 1 in accordance with an embodiment of the present disclosure. For example, a research organization A wants to experiment a newly developed experimental drug Z 130 for COVID 19. According to developmental research, the experimental drug Z 130 enhances immunity for diabetic patients above the age of 65.

First, the platform 10 via a clinical trial registration module 20 registers a number of patients. The patients mainly provide at least one of name, age, contact details, address, medical history, family history, genetic data, nutritional data or combination thereof. The clinical trial registration module 20 categorizes the patient group X 120, whereby each patient is above the age of 65 and additionally suffers from diabetes.

After such categorization, the platform 10 via a medical compliance module 30 starts monitoring each patient of the patient group X 120. The newly developed experimental drug Z 130 for COVID 19 is administered to each patient of the group X 120 with time specific injection dosage. The medical compliance module 30 is real time captures all bio-physical parameters, discomfort of each drug administered patients, side affects and the like. Such details are stored vi a database management module 40.

The medical compliance module 30 also helps in enforcing complete adherence of treatment plan corresponding to the experimental drug Z 130. An adherence report 140 as well as health report 140 are generated on timely basis. Each such report 140 is stored via the database management report 40. It is pertinent to note the stored report 140 may be classified according to privacy level to provide a security to research.

Further, in such exemplary embodiment, another similar research organization B may be doing clinical trial targeting the same age group and same comorbidity. The database management module 40 may enable storing of the research documents and adherence report of each patient of such trials also in the platform 10.

It is pertinent to note that the medical compliance module 40 enables proper mapping of each clinical organization reports for better understanding. Such mapping helps in fast and easy research and development. Further, via an access control module 50 each research organization may cross examine each other specific privacy level reports 140 and do collaborative clinical trial.

The clinical trial registration module 20, the medical compliance module 30, the database management module 40 and the access control module 50 in FIG. 2 is substantially equivalent to the clinical trial registration module 20, the medical compliance module 30, the database management module 40 and the access control module 50 of FIG. 1.

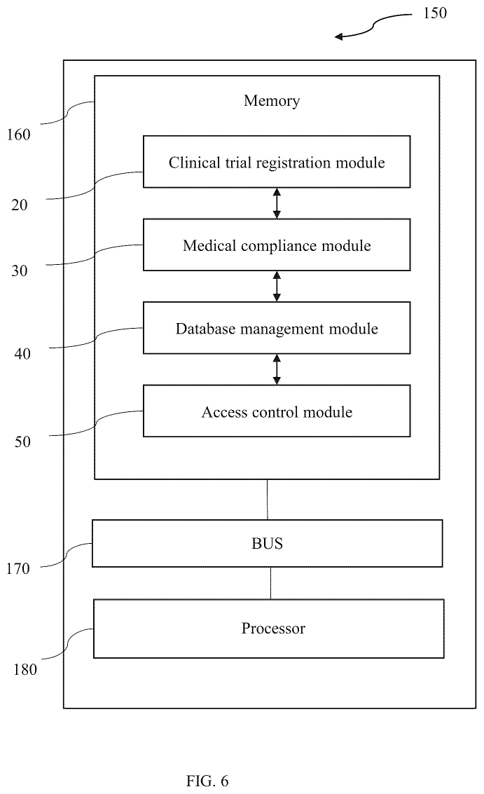

FIG. 6 is a block diagram of a computer or a server 150 in accordance with an embodiment of the present disclosure. The server 150 includes processor(s) 180, and memory 160 coupled to the processor(s) 180.

The processor(s) 180, as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory 160 includes a plurality of modules stored in the form of executable program which instructs the processor 180 via a bus 170 to perform the method steps illustrated in FIG. 1. The memory 160 has following modules: the clinical trial registration module 20, the medical compliance module 30, the database management module 40 and the access control module 50.

The clinical trial registration module 20 is configured to on-board one or more patients for the clinical trial for one or more clinical research organizations based at least one of name, age, contact details, address, and medical history or combination thereof. The clinical trial registration module 20 is also configured to categorise on-boarded one or more patients based on age and health condition.

The medical compliance module 30 is configured to capture data representative of at least one of one or more diseases, one or more bio-physical parameters indicative of health condition of the one or more patients, type of discomfort, severity of discomfort, part of body experiencing the discomfort, time of discomfort, one or more treatment plans designed for the clinical trials, one or more medications prescribed in accordance with the one or more treatment plans, one or more symptoms, one or more medication side-effects via communicatively coupled one or more devices associated with at least one of each of the one or more patients, one or more healthcare personnel and one or more doctors.

The medical compliance module 30 is also configured to monitor adherence of each of the one or more patients to the one or more treatment plans designed for the clinical trials, prescribed follow-ups and actions. The medical compliance module 30 is also configured to generate a medical adherence report corresponding to each of the one or more patients in compliance with the one or more treatment plans designed for the clinical trials.

The database management module 40 is configured to automatically store data captured by the medical compliance module and generated medical adherence report for each of the one or more patients. The database management module 40 is also configured to receive and store data associated with the clinical trial from the one or more clinical research organisations based on one of data migration, data conversion and data integration techniques. The database management module 40 is also configured to classify stored data associated with the clinical trial based on predesigned privacy levels.

The access control module 50 is configured to provide access to a specific privacy level of classified stored data associated with the clinical trial corresponding to the one or more clinical research organisations based on a permitted access to a specific privacy level. The access control module 50 is configured to enable collaborative clinical trial among the one or more clinical research organisations by sharing the data associated with the clinical trial in real-time.

Computer memory elements may include any suitable memory device(s) for storing data and executable program, such as read only memory, random access memory, erasable programmable read only memory, electrically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the processor(s) 180.

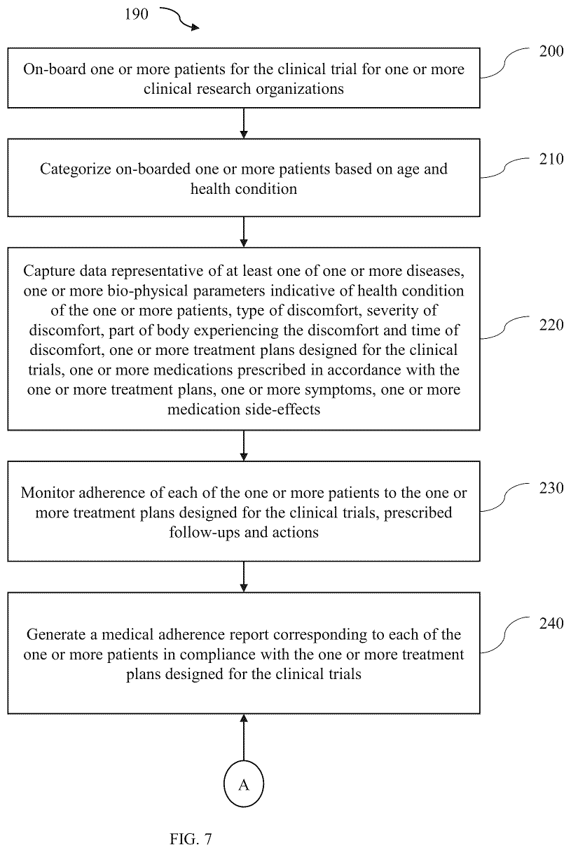

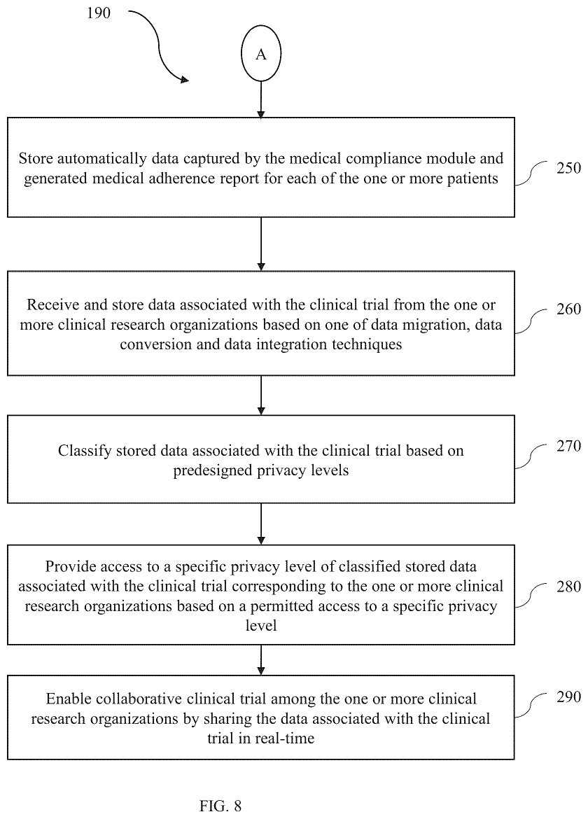

FIG. 7 is a flowchart representing the steps of a method 190 for managing a clinical trial on a SaaS platform in accordance with an embodiment of the present disclosure. FIG. 8 is a flowchart representing the steps of a method 190 for managing a clinical trial on a SaaS platform in accordance with an embodiment of the present disclosure.

The method 190 includes on-boarding one or more patients for the clinical trial for one or more clinical research organizations based at least one of name, age, contact details, address, and medical history or combination thereof in step 200. In one embodiment, on-boarding the one or more patients for the clinical trial for the one or more clinical research organizations based at least one of name, age, contact details, address, and medical history or combination thereof includes on-boarding the one or more patients for the clinical trial for the one or more clinical research organizations by a clinical trial registration module. In another embodiment, on-boarding the one or more patients for the clinical trial for the one or more clinical research organizations based at least one of name, age, contact details, address, and medical history or combination thereof includes on-boarding the one or more patients for the clinical trial for the one or more clinical research organizations comprising government agencies, private agencies, pharmaceutical companies, and institutes.

The method 190 also includes categorizing on-boarded one or more patients based on age and health condition in step 210. In one embodiment, categorizing the on-boarded one or more patients based on age and health condition includes categorizing the on-boarded one or more patients by the clinical trial registration module.

The method 190 also includes capturing data representative of at least one of one or more diseases, one or more bio-physical parameters indicative of health condition of the one or more patients, type of discomfort, severity of discomfort, part of body experiencing the discomfort and time of discomfort, one or more treatment plans designed for the clinical trials, one or more medications prescribed in accordance with the one or more treatment plans, one or more symptoms, one or more medication side-effects via communicatively coupled one or more devices associated with at least one of each of the one or more patients, one or more healthcare personnel and one or more doctors in step 220. In one embodiment, capturing the data representative of at least one of one or more diseases includes capturing the data representative of at least one of one or more diseases by a medical compliance module.

The method 190 also includes monitoring adherence of each of the one or more patients to the one or more treatment plans designed for the clinical trials, prescribed follow-ups and actions in step 230. In one embodiment, monitoring the adherence of each of the one or more patients to the one or more treatment plans designed for the clinical trials, prescribed follow-ups and actions includes monitoring adherence of each of the one or more patients to the one or more treatment plans designed for the clinical trials, prescribed follow-ups and actions by the medical compliance module.

The method 190 also includes generating a medical adherence report corresponding to each of the one or more patients in compliance with the one or more treatment plans designed for the clinical trials in step 240. In one embodiment, generating the medical adherence report corresponding to each of the one or more patients in compliance with the one or more treatment plans designed for the clinical trials includes generating the medical adherence report corresponding to each of the one or more patients in compliance with the one or more treatment plans designed for the clinical trials by the medical compliance module.

The method 190 also includes monitoring the one or more treatment plans by the medical compliance module. In one embodiment, monitoring the one or more treatment plans includes mapping the one or more treatment plans generated by the one or more clinical research organisation with the one or more treatment plans generated by another clinical research organisation. In another embodiment, monitoring the one or more treatment plans includes identifying differences in at least one of medications, prescribed follow-ups and actions, one or more symptoms, one or more medication side-effects and health status improvement rate.

In yet another embodiment, monitoring the one or more treatment plans includes suggesting standardisation of the clinical trial parameters based on identified differences in at least one of medications, prescribed follow-ups and actions, one or more symptoms, one or more medication side-effects and health status improvement rate. In one embodiment, monitoring the one or more treatment plans includes monitoring adherence of each of the one or more patients to the one or more treatment plans designed for the clinical trials, prescribed follow-ups and actions at a micro level. In such embodiment, monitoring adherence of each of the one or more patients includes monitoring at the micro level comprises monitoring daily medication intakes in accordance with the one or more treatment plans, monitoring follow-up treatment compliance in accordance with the one or more treatment plans, monitoring medical appointments in accordance with the one or more treatment plans and monitoring ongoing symptoms and side-effects.

In one embodiment, monitoring the one or more treatment plans includes comparing end results of the one or more clinical trials. In one such embodiment, comparing end results of the one or more clinical trials includes comparing blind controls or double-blind randomization with the end results of an ongoing clinical trials. In another such embodiment, comparing the end results of the one or more clinical trials includes comparing analysis of different clinical trials for same disease or medications.

The method 190 also includes storing automatically data captured by the medical compliance module and generated medical adherence report for each of the one or more patients in step 250. In one embodiment, storing automatically the data captured by the medical compliance module and the generated medical adherence report for each of the one or more patients includes storing automatically data captured by the medical compliance module and generated medical adherence report for each of the one or more patients by a database management module.

The method 190 also includes receiving and storing data associated with the clinical trial from the one or more clinical research organisations based on one of data migration, data conversion and data integration techniques in step 260. In one embodiment, receiving and storing the data associated with the clinical trial from the one or more clinical research organisations based on one of the data migration, the data conversion and the data integration techniques includes receiving and storing the data associated with the clinical trial from the one or more clinical research organisations by the database management module.

In another embodiment, receiving and storing the data associated with the clinical trial from the one or more clinical research organisations based on one of the data migration, the data conversion and the data integration techniques includes receiving and storing data associated with the clinical trial from the one or more clinical research organisations comprising data migration such as migrating databases, migrating applications and migrating to the cloud.

The method 190 also includes classifying stored data associated with the clinical trial based on predesigned privacy levels instep 270. In one embodiment, classifying the stored data associated with the clinical trial based on the predesigned privacy levels includes classifying the stored data associated with the clinical trial based on the predesigned privacy levels by the database management module.

The method 190 also includes providing access to a specific privacy level of classified stored data associated with the clinical trial corresponding to the one or more clinical research organisations based on a permitted access to a specific privacy level in step 280. In one embodiment, providing access to the specific privacy level of the classified stored data associated with the clinical trial corresponding to the one or more clinical research organisations based on the permitted access to a specific privacy level includes providing access to the specific privacy level of the classified stored data associated with the clinical trial corresponding to the one or more clinical research organisations based on the permitted access to a specific privacy level by an access control module.

The method 190 also includes enabling collaborative clinical trial among the one or more clinical research organisations by sharing the data associated with the clinical trial in real-time in step 290. In one embodiment, enabling the collaborative clinical trial among the one or more clinical research organisations by sharing the data associated with the clinical trial in real-time includes enabling the collaborative clinical trial among the one or more clinical research organisations by sharing the data associated with the clinical trial in real-time by the access control module.

The method 190 also includes facilitating exchange of real-time feedbacks among the one or more clinical research organisations. In one embodiment, facilitating the exchange of real-time feedbacks among the one or more clinical research organisations includes facilitating the exchange of real-time feedbacks among the one or more clinical research organisations by the feedback module. In such embodiment, facilitating the exchange of real-time feedbacks among the one or more clinical research organisations includes enable constant monitoring of each of the one or more patient's health condition.

Present disclosure of a SaaS platform enables real time managing of one or more clinical trials associated with multiple drugs. The platform completely automates the tracking and monitoring procedures of any clinical trial. Additionally, the platform enables categorization of groups in clinical trial to facilitate more specific target-oriented drug. The platform also enables time to time adherence checking of the clinical trial treatment plan. Whereby the platform provides mass scale enablement of remote care.

The platform facilitates sharing of data within multiple research organizations with privacy level to fast track any particular research. Thereby, increasing the output for research and development and mass surveillance of illness without compromising patient's consent and privacy. By virtue of this, the research institutions need not to put all the patients undergoing clinical trials at one place. This greatly helps in utilising the resources scattered over multiple geolocations, thus making the system robust. The resources may include, but not limited to, beds, medical equipment, doctors, and caregivers.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependant on other acts may be performed in parallel with the other acts. The scope of embodiments is by no means limited by these specific examples.

We claim:

1. A SaaS platform to manage a clinical trial, comprising:
a clinical trial registration module hosted in a presentation tier of the SaaS platform, wherein the clinical trial registration module is configured to:
on-board one or more patients for the clinical trial for one or more clinical research organizations based at least one of name, age, contact details, address, medical history, family history, genetic data, nutritional data or combination thereof, and
categorise on-boarded one or more patients based on age and health condition;
a medical compliance module hosted in a logic tier of the SaaS platform, wherein the medical compliance module is configured to:
capture data representative of at least one of one or more diseases, one or more bio-physical parameters indicative of health condition of the one or more patients, type of discomfort, severity of discomfort, part of body experiencing the discomfort, time of discomfort, one or more treatment plans designed for the clinical trials, one or more medications prescribed in accordance with the one or more treatment plans, one or more symptoms, one or more medication side-effects via communicatively coupled one or more devices associated with at least one of each of the one or more patients, one or more healthcare personnel and one or more doctors;
monitor adherence of each of the one or more patients to the one or more treatment plans designed for the clinical trials, prescribed follow-ups and actions; and
generate a medical adherence report corresponding to each of the one or more patients in compliance with the one or more treatment plans designed for the clinical trials;
a database management module hosted in a data tier of the SaaS platform, wherein the database management module is configured to:
automatically store data captured by the medical compliance module and generated medical adherence report for each of the one or more patients;
receive and store data associated with the clinical trial from the one or more clinical research organisations based on one of data migration, data conversion and data integration techniques; and
classify stored data associated with the clinical trial based on predesigned privacy levels; and
an access control module hosted in the in the logic tier of the SaaS platform, wherein the access control module is configured to:
provide access to a specific privacy level of classified stored data associated with the clinical trial corresponding to the one or more clinical research organisations based on a permitted access to a specific privacy level; and
enable collaborative clinical trial among the one or more clinical research organisations by sharing the data associated with the clinical trial in real-time.

2. The SaaS platform as claimed in claim 1, wherein the SaaS platform comprises one of a multi-tenant SaaS platform and single-tenant SaaS platform.

3. The SaaS platform as claimed in claim 1, wherein the data migration comprises migrating databases, migrating applications and migrating to the cloud.

4. The SaaS platform as claimed in claim 1, wherein the one or more clinical research organizations comprise government agencies, private agencies, pharmaceutical companies, and institutes.

5. The SaaS platform as claimed in claim 1, wherein the medical compliance module is also configured to:
map the one or more treatment plans generated by the one or more clinical research organisation with the one or more treatment plans generated by another clinical research organisation;
identify differences in at least one of medications, prescribed follow-ups and actions, one or more symptoms, one or more medication side-effects and health status improvement rate; and
suggest standardisation of the clinical trial parameters based on identified differences in at least one of medications, prescribed follow-ups and actions, one or more symptoms, one or more medication side-effects and health status improvement rate.

6. The SaaS platform as claimed in claim 1, wherein the medical compliance module is also configured to compare end results of the one or more clinical trials.

7. The SaaS platform as claimed in claim 6, wherein the medical compliance module compares blind controls or double-blind randomization with the end results of an ongoing clinical trials and compares analysis of different clinical trials for same disease or medications.

8. The SaaS platform as claimed in claim 1, wherein monitor adherence of each of the one or more patients to the one or more treatment plans designed for the clinical trials, prescribed follow-ups and actions at a micro level,
wherein the adherence at the micro level comprises monitoring daily medication intakes in accordance with the one or more treatment plans, monitoring follow-up treatment compliance in accordance with the one or more treatment plans, monitoring medical appointments in accordance with the one or more treatment plans and monitoring ongoing symptoms and side-effects.

9. The SaaS platform as claimed in claim 1, comprising a feedback module hosted in the in the data tier of the SaaS platform, wherein the feedback module is configured to facilitate exchange of real-time feedbacks among the one or more clinical research organisations, wherein the real-time feedbacks enable constant monitoring of each of the one or more patients health condition.

10. A method for managing a clinical trial on a SaaS platform, the method comprising:
on-boarding, by a clinical trial registration module, one or more patients for the clinical trial for one or more clinical research organizations based at least one of name, age, contact details, address, medical history, family history, genetic data, nutritional data or combination thereof;
categorizing, by the clinical trial registration module, on-boarded one or more patients based on age and health condition;
capturing, by a medical compliance module, data representative of at least one of one or more diseases, one or more bio-physical parameters indicative of health condition of the one or more patients, type of discomfort, severity of discomfort, part of body experiencing the discomfort and time of discomfort, one or more treatment plans designed for the clinical trials, one or more medications prescribed in accordance with the one or more treatment plans, one or more symptoms, one or more medication side-effects via communicatively coupled one or more devices associated with at least one of each of the one or more patients, one or more healthcare personnel and one or more doctors;
monitoring, by the medical compliance module, adherence of each of the one or more patients to the one or more treatment plans designed for the clinical trials, prescribed follow-ups and actions;
generating, by the medical compliance module, a medical adherence report corresponding to each of the one or more patients in compliance with the one or more treatment plans designed for the clinical trials;
storing, by a database management module, automatically data captured by the medical compliance module and generated medical adherence report for each of the one or more patients;
receiving and storing, by the database management module, data associated with the clinical trial from the one or more clinical research organisations based on one of data migration, data conversion and data integration techniques;
classifying, by the database management module, stored data associated with the clinical trial based on predesigned privacy levels;
providing, by an access control module, access to a specific privacy level of classified stored data associated with the clinical trial corresponding to the one or more clinical research organisations based on a permitted access to a specific privacy level; and
enabling, by the access control module, collaborative clinical trial among the one or more clinical research organisations by sharing the data associated with the clinical trial in real-time.

11. The method as claimed in claim 10, wherein on-boarding, by a clinical trial registration module, the one or more patients for the clinical trial for the one or more clinical research organizations comprising government agencies, private agencies, pharmaceutical companies, and institutes.

12. The method as claimed in claim 10, wherein receiving and storing, by the database management module, data associated with the clinical trial from the one or more clinical research organisations comprises data migration such as migrating databases, migrating applications and migrating to the cloud.

13. The method as claimed in claim 10, wherein monitoring, by the medical compliance module, the one or more treatment plans comprises
mapping the one or more treatment plans generated by the one or more clinical research organisation with the one or more treatment plans generated by another clinical research organisation;
identifying differences in at least one of medications, prescribed follow-ups and actions, one or more symptoms, one or more medication side-effects and health status improvement rate; and
suggesting standardisation of the clinical trial parameters based on identified differences in at least one of medications, prescribed follow-ups and actions, one or more symptoms, one or more medication side-effects and health status improvement rate.

14. The method as claimed in claim 10, wherein monitoring, by the medical compliance module, the one or more treatment plans comprises comparing end results of the one or more clinical trials, wherein comparing end results of the one or more clinical trials comprises
comparing blind controls or double-blind randomization with the end results of an ongoing clinical trials; and
comparing analysis of different clinical trials for same disease or medications.

15. The method as claimed in claim 10, wherein monitoring, by the medical compliance module, adherence of each of the one or more patients to the one or more treatment plans designed for the clinical trials, prescribed follow-ups and actions at a micro level.

16. The method as claimed in claim 15, wherein monitoring, by the medical compliance module, at the micro level comprises monitoring daily medication intakes in accordance with the one or more treatment plans, monitoring follow-up treatment compliance in accordance with the one or more treatment plans, monitoring medical appointments in accordance with the one or more treatment plans and monitoring ongoing symptoms and side-effects.

17. The method as claimed in claim 10, comprising facilitating, by the feedback module, exchange of real-time feedbacks among the one or more clinical research organisations, wherein the real-time feedbacks enable constant monitoring of each of the one or more patients health condition.
